(19) United States
(12) Patent Application Publication
(10) Pub. No.: US 2026/0256827 A1
(43) Pub. Date: Sep. 3, 2026

(54) Methods for treating lower back pain with hyaluronic acid.

(71) Applicant: David Donaldson Brown, Placentia, CA (US)

(72) Inventor: David Donaldson Brown, Placentia, CA (US)

(21) Appl. No.: 19/654,906

(22) Filed: Apr. 22, 2026

Related U.S. Application Data
(63) Continuation of application No. PCT/US24/53392, filed on Oct. 29, 2024.
(60) Provisional application No. 63/594,020, filed on Oct. 29, 2023.

(51) Int. Cl. A61K 31/728; A61K 9/00; A61P 29/00

(52) U.S. Cl. CPC A61K 31/728; A61K 9/0019; A61P 29/00

(57) ABSTRACT

The present invention provides methods for treating lower back, buttocks, and/or sacral region pain in a subject, comprising administering to the subject, by one or more injections, a composition comprising hyaluronic acid (HA) and a pharmaceutically acceptable carrier. The injections are made into, or immediately adjacent to, soft tissue attachments to the subject's iliac crest, posterior superior iliac spine, and/or sacrum.

### CROSS-REFERENCE TO RELATED APPLICATIONS

This application is a continuation of International Application No. PCT/US2024/053392, filed on Oct. 29, 2024, which claims priority to U.S. Provisional Application No. 63/594,020, filed on Oct. 29, 2023, the contents of each of which are herein incorporated by reference in their entirety.

### BACKGROUND

Chronic lower back pain is a widespread problem, affecting a significant percentage of the population. Most chronic lower back pain is considered nonspecific in origin.¹ Nevertheless, certain anatomical structures are generally recognized as pain generators in a certain fraction of individuals with chronic low back pain, including the lumbar facet joints, intervertebral discs, and sacroiliac joint. Other sources of lower back pain include myofascial pain, arising from the muscles or their fascial coverings (to be distinguished from tendinopathy or enthesopathy); rare spondyloarthropathies including ankylosing spondylitis and psoriatic arthritis, which are characterized by enthesitis (inflammation of the attachment sites of tendons/ligaments to bone); and entrapment of the superior or middle cluneal nerves as they cross the iliac crest, which is associated with paresthesia, a characteristic feature of neuropathic pain.²⁻⁴

It is frequently difficult to treat effectively, and existing treatments either work in only a limited percentage of individuals, are only modestly effective, or have considerable potential side effects, including risks of drug addiction. Nonsteroidal anti-inflammatory medications are commonly used, including agents such as naproxen, meloxicam, celecoxib, amidst others, but this class of medications only demonstrates modest effectiveness in clinical practice and in controlled clinical trials. Muscle relaxants such as baclofen, cyclobenzaprine, carisoprodol and others may be helpful in anecdotal cases of lower back pain, but they have less than clear benefit in controlled trials.⁵ Opioid class pain medications are also widely used to treat chronic lower back pain, but clinical evidence is limited to short-term studies, with modest effects.⁵ In addition, there are well known risks of addiction and abuse with this class of medication. Therefore, a need exists for new methods for treating lower back pain that demonstrate therapeutic efficacy, with reduced potential for deleterious side effects.

### SUMMARY

In an aspect, the present invention provides methods for treating lower back, buttocks, and/or sacral region pain in a subject. The methods comprise administering to the subject, by one or more injections, a composition comprising a therapeutically effective amount of hyaluronic acid (HA) and a pharmaceutically acceptable carrier. The injections are made into, or immediately adjacent to, soft tissue attachments to the subject's iliac crest, posterior superior iliac spine, and/or sacrum. The pain may be nociceptive pain, and the methods may further comprise palpating the iliac crest, posterior superior iliac spine, and/or sacrum and selecting areas of pain/tenderness for injection.

### BRIEF DESCRIPTION OF THE DRAWINGS

FIG. 1 is a drawing illustrating the location of the sacrum [100] with respect to recognized surface landmarks and specifically identifies locations on the sacrum to be palpated for tenderness and optionally injected with hyaluronic acid using “X” as a marker to identify the locations relative to the left side of the subject. Sites for palpation and optional hyaluronic acid injection are located immediately lateral to the median crest of the sacrum [101] and along the lateral edge/margin of the sacrum [102]. Four sites for palpation and optional hyaluronic acid injection are marked at each location.

FIG. 2 is a drawing illustrating the origin of the gluteus maximus [201] along the lateral edge/margin of the sacrum [102] and posterior superior iliac spine [202], with the “X” marking sites of palpation and optional injection. Also depicted is the origin of the gluteus medius [203] along the inferior margin of the iliac crest [204], another site that may be palpated and injected with hyaluronic acid as shown. Referring to location [102] along the lateral edge/margin of the sacrum, four sites for palpation and optional hyaluronic acid injection are marked. Referring to location [202] along the lateral margin of the posterior superior iliac spine, two sites for palpation and optional hyaluronic acid injection are marked. Referring to location [204] along the inferior margin of the iliac crest, four sites for palpation and optional hyaluronic acid injection are marked.

FIG. 3 is a drawing illustrating the medial and superior margins of the posterior superior iliac spine [303] and the superior margin of the iliac crest [302], where the thoracolumbar fascia, specifically the interwoven thoracolumbar composite [301] attaches. Sites marked with an “X” are palpated for tenderness and optionally injected with hyaluronic acid. Referring to location along the medial and superior margins of the posterior superior iliac spine, two sites for palpation and optional hyaluronic acid injection are marked. Referring to location [302] along the superior margin of the iliac crest, four sites for palpation and optional hyaluronic acid injection are marked.

FIG. 4 is a drawing illustrating the complex underlying ligamentous anatomy of the sacrum, including the posterior sacroiliac ligaments [401] and the sacrotuberous ligament [402]. Sacral ligaments are present deep into the muscle layer, making it difficult to differentiate between pain arising from tendinous, aponeurotic, fascial, or ligamentous attachments. Thus, the pain treated by the present invention may be of ligamentous, tendinous, aponeurotic, or myofascial origin, or may be caused by periostitis of the iliac crest or sacrum.

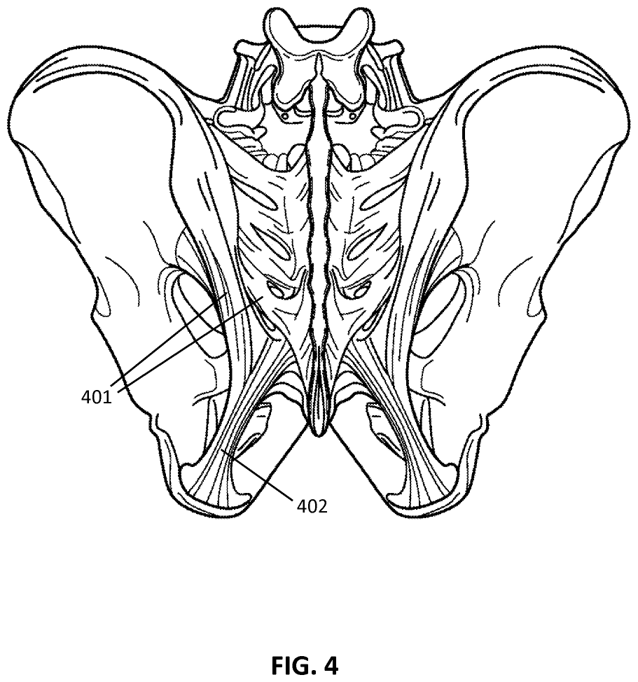

### DETAILED DESCRIPTION

Several studies have identified the soft attachments to the iliac crest as a potential source of chronic back pain. For example, reports have described (1) treatment of “iliac crest pain syndrome” as the purported pain source in approximately 40% of individuals with undifferentiated chronic lower back pain by local anesthetic injection^{6-7,18}, (2) an “iliolumbar syndrome”, characterized by lower back pain arising from attachments of the iliolumbar ligament to the iliac crest⁸⁻⁹, (3) thickening of the erector spinae entheses in lower back pain¹⁰, and (4) treatment of what are presumed to be iliac crest enthesopathies by injection either local anesthetic or prolotherapy in patients with persistent back pain following lumbar spine surgery and had persistent lower back pain¹¹. However, no pathological studies that include biopsy evaluation have investigated the contribution of tissue impairment of the soft tissue attachments to the iliac crest or sacrum in patients with common chronic lower back or buttocks pain.

Hyaluronic acid (HA) is a large, naturally occurring polymer that is a major constituent of synovial fluid, tendon, ligament and extracellular matrix and is involved in lubricating joint movements. In addition to its lubricating properties, HA exhibits anti-inflammatory properties that are dependent on concentration and molecular weight.¹²

HA is FDA-approved as an injection treatment for osteoarthritis of the knee, where the therapeutic effect has been believed to be mediated primarily by its lubricant properties. In addition, HA has been investigated extensively for its use in other arthritic joints. While HA has been used with some degree of success in several tendinous disorders, including supraspinatus tendinosis, lateral epicondylitis, and patellar tendinopathy, no evidence has demonstrated the efficacy of HA in treating ligamentous enthesopathies.¹³ Several attempts at using HA to treat lower back pain have been reported.¹⁴⁻¹⁵ However, these studies have produced mixed results, and have been limited to investigating the effect of HA on pain associated with (1) the fascial planes of the thoracolumbar fascia, (2) neuropathic origin (e.g., lumbar radiculopathy), (3) degenerative disc disease, or (4) vertebral facet joint arthritis.

As demonstrated in the Examples, the present inventor discovered that injection of HA into the soft tissue attachments to the iliac crest, the posterior superior iliac spine, and/or the sacrum was effective in treating patients with lower back, sacral, and/or buttocks pain. Thus, the present invention provides methods for treating pain comprising administering one or more HA injections.

### Lower Back, Buttocks, and or Sacral Pain

In an aspect, the present invention provides methods for treating pain in a subject, wherein the subject is experiencing pain in the lower back, buttocks, and/or sacral region. A “subject” or “patient” refers to any organism in need of and/or subjected to a treatment, such as a farm animal, domestic animal, or human. In some embodiments, the subject is a human. As used herein, “lower back” refers to the lumbar spine region between the last thoracic vertebra (T12) and the first sacral vertebra (S1) and extending laterally to include the iliac crests, “sacral area” refers to the region at the base of the spine which comprises the sacrum, and “buttocks” refers to the region below the lower back, including all soft tissue and bone extending to the gluteal sulcus on each side.

In an aspect, the methods comprise administering to the subject a composition comprising a therapeutically effective amount of HA. HA is a polymer of D-glucuronic acid and N-acetyl-D-glucosamine that occurs naturally at varied sizes, with molecular weights ranging from 5 to 20,000 kilodaltons (kDa). The present methods may use HA having any suitable molecular weight. In some embodiments, the composition comprises HA having a molecular weight of about 50, 75, 100, 200, 300, 400, 500, 600, 700, 800, 900, 1000, 5000, 10000, 15000, or 20000 kDa, or a molecular weight within a range bounded by any of the forgoing. In some embodiments, the HA has a molecular weight of at least 500 kDa. In some embodiments, the HA has a molecular weight of about 500 kDa to about 730 kDa, or between about 450 kDa and 800 kDa, or between 400 kDa and 1000 kDa. The HA may be present in the composition at any suitable concentration. In some embodiments, the HA is present in the composition at a concentration of about 1 mg/mL, 5 mg/mL, 10 mg/mL, 15 mg/mL, 20 mg/mL, or 25 mg/mL, or at a concentration within a range bounded by any of the foregoing. In some embodiments, the HA is present in the composition at a concentration of about 10 mg/mL. The concentration of HA in the composition may be between 9-11mg/mL, between 8-12 mg/mL or between 5 -15 mg/mL.

In an aspect, the composition further comprises a pharmaceutically acceptable carrier. Pharmaceutically acceptable carriers are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, and nanoparticles. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, or suspensions, including saline and buffered media. The composition may further comprise additives such as albumin or gelatin to prevent absorption to surfaces, detergents (e.g., Tween 20, Tween 80, Pluronic F68, bile acid salts), anti-oxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels, etc.) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

In some embodiments, the pharmaceutically acceptable carrier comprises sodium chloride, monobasic sodium phosphate, dibasic sodium phosphate, and water. HA is FDA-approved and commercially available as an injection treatment for osteoarthritis of the knee in several brands of differing molecular weights (including, without limitation, Hyalgan, Synvisc™, Monovisc™, Orthovisc™, Durolane™). Thus, in some embodiments, the methods comprise administering a commercially available formulation,

As used herein, the term “therapeutically effective amount” refers to an amount of HA sufficient to treat pain in a subject receiving the composition. Methods for assessing pain treatment are known in the art and include, without limitation, assessments based on pain severity (average and/or maximum) and pain duration. In some embodiments, pain severity is assessed using a Visual analog numerical rating scale ranging from 0 (no pain) to 10 (worst pain possible). Average and maximum pain severity may be assessed on a daily, weekly, or monthly basis. In some embodiments, the patient's average pain severity and/or maximum pain severity is reduced following administration of the HA composition. In some embodiments, the total number of hours per day during which the patient experiences pain is reduced following administration of the HA composition. In some embodiments, the number of pain-free days per week and/or month is increased following administration of the HA composition.

The HA composition may be administered to a patient experiencing acute pain or chronic pain. As used herein, “chronic pain” refers to pain that has persisted for a duration of at least 3 months, while “acute pain” refers to sudden, temporary pain that typically resolves in less than 3 months. The HA composition may be administered to a patient experiencing pain of any severity, In some embodiments, the HA composition is administered to a patient experiencing chronic lower back, buttocks, and/or sacral region pain, with a daily maximum pain severity of at least 7/10 on the visual analog numerical rating scale.

In some embodiments, the pain is nociceptive pain. As used herein, “nociceptive pain” refers to pain caused by/arising from damage to body tissue(s), Nociceptive pain is distinguished from neuropathic pain, which is caused by damage to the nervous system. In some embodiments, the nociceptive pain arises from the soft tissues immediately along the iliac crest, posterior superior iliac spine, and/or sacrum, As used herein, “soft tissues” refer to supportive and/or connective body tissues, including muscles, tendons, ligaments, and aponeuroses. In some embodiments, the source of pain in the soft tissues is an enthesopathy affecting the ligamentous, tendinous and aponeurotic attachments of the lower back and buttocks to the iliac crest, posterior superior iliac spine, and/or to the posterior and lateral sacral area. The source of pain in the soft tissues may also be of myofascial origin (originating from muscles or their fascial covering), or may be caused by periostitis (inflammation of the periosteum surrounding bones) of the iliac crest or sacrum.

In some embodiments, the subject has one or more co-morbid source of lower back, buttocks, and/or sacral pain, such as degenerative disc disease, facet joint arthritis, sacroiliac joint arthritis, osteoporotic vertebral compression fracture, lumbar radiculopathy, or spinal stenosis. A skilled artisan will readily appreciate the available methods for correctly diagnosing these other conditions. In other embodiments, the composition is administered to a subject who does not have additional co-morbid conditions contributing to the pain. These separate sources of lower back pain may be excluded by appropriate history, examination and diagnostic studies. For example, to exclude a peripheral neuropathic origin of pain in these areas, such as from cluneal neuralgia or neuropathy, there should be no associated paresthesia or sensory loss.

In an aspect, the composition is administered by one or more injections. The composition may be administered by 1, 2, 3, 4, 5, 6, 7, 8, 9, 10, 15, 20, 25, or 30 injections, or by a number of injections within a range bounded by any of the foregoing. The injections may be repeated every 4-8 weeks indefinitely, or may be administered on an as needed basis indefinitely. In some embodiments, the composition is administered by 2-10 injections. The injections may be made using any suitable needle, including, without limitation, a 30 gauge, ½″ needle, and may deliver any suitable amount of HA. In some embodiments, each injection delivers about 0.25, 0.5, 0.75, 1, 1.5, 2, 2.5, 3, 4, 5, 6, 7, 8, 9, or 10 mL of the composition, or a volume of the composition within a range bounded by any of the foregoing. In some embodiments, each injection delivers about 0.5 mL to 1 mL of the composition. In some embodiments, each injection delivers 1, 2, 3, 4, 5, 6, 7, 8, 9, 10, 20, 30, 40, or 50 mg of HA, or an amount of HA within a range bounded by any of the foregoing. In some embodiments, each injection delivers 5 mg or more of HA. In some embodiments, each injection delivers 5 mg to 10 mg of HA. The amount of HA delivered may be independently selected for each injection.

In some embodiments, the injections are made into, or immediately adjacent to, the soft tissue attachments to the subject's iliac crest, posterior superior iliac spine, and/or sacrum. The injections may be made within about ½″ to about 1″ from the iliac crest, posterior superior iliac spine, and/or sacrum. Injections may acceptably be made into soft tissue immediately adjacent to the bony attachments of tendon or ligament to the palpated locations, as long as the injected volume of hyaluronic acid is sufficient to spread to these attachments. Larger injection volumes and dosages may allow for injection at greater distances from these locations, but injections into or immediately adjacent to such distant structures as the spinal canal, neural foramina, intervertebral discs, intrafascial planes, or facet joints are not contemplated by these methods. Within the specified locations and distances to perform injections, the thoracolumbar composite has formed, which is an interwoven layer of previously separated fascial layers of the thoracolumbar fascia, making it impossible to inject hyaluronic acid between fascial layers. at these specified locations.¹⁷

In some embodiments, the composition is administered by two or more injections, spaced at an interval. The two or more injections may be spaced at an interval of about ¼″, ½″, ¾″, 1″, 1 ¼″, 1 ½″, 1 ¾″, 2″, 3″, or 4″, or by an interval within a range bounded by any of the foregoing. In some embodiments, the two or more injections are spaced at an interval of about ½″ to about 1.″

In some embodiments, the methods further comprise identifying one or more injection sites for the one or more injections, comprising palpating the iliac crest, posterior superior iliac spine, and/or sacrum. As used herein, “palpating” or “palpate” refers to a manual examination designed to assess by touch/feel one or more of the size, shape, firmness, location, consistency, texture, and/or tenderness of a body part. The palpation may be used to detect focal tenderness on or along the sacrum, posterior superior iliac spine, and/or iliac crest of one or more of the following the tendonous attachments (entheses): the erector spinae, multifidus, gluteus maximus, gluteus medius, quadratus lumborum, and/or external and internal oblique muscles. The focal tenderness may be identified when the subject vocalizes pain, winces, or move their body in response to the pain elicited by firm palpation, and may reveal pain arising from tendinous attachments, ligamentous attachments, or periosteum.

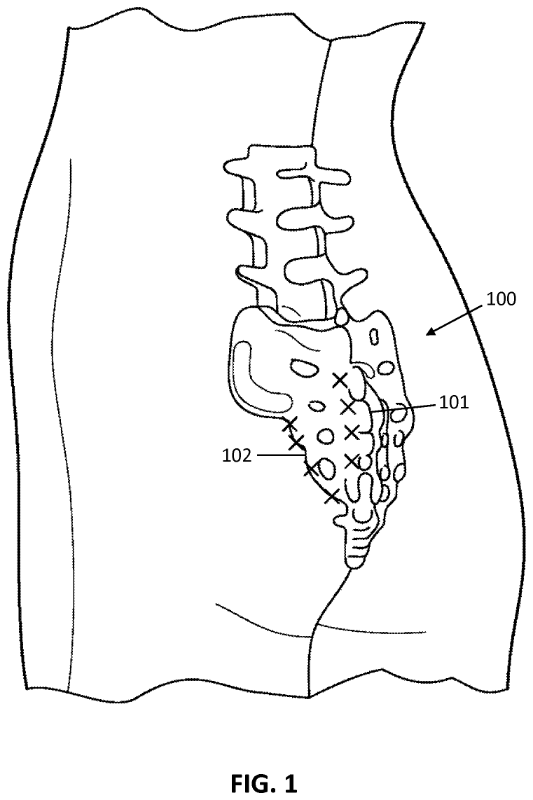

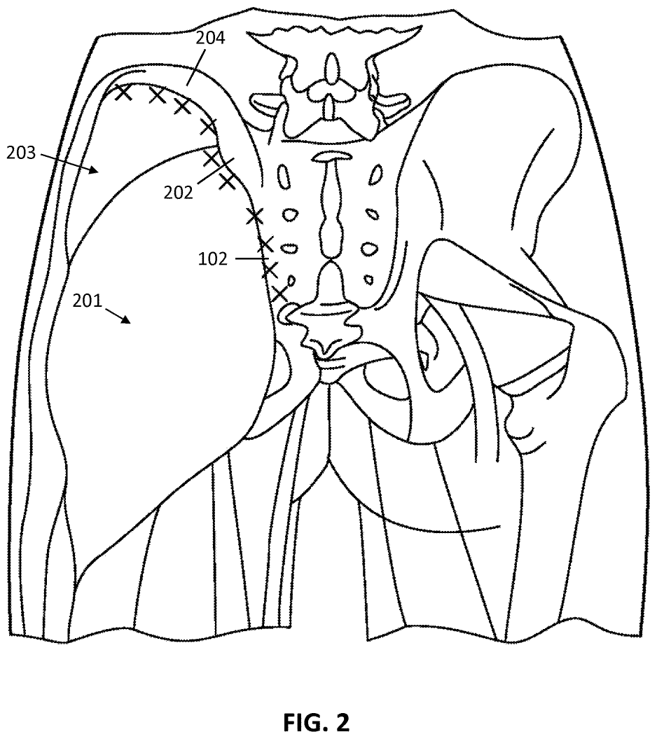

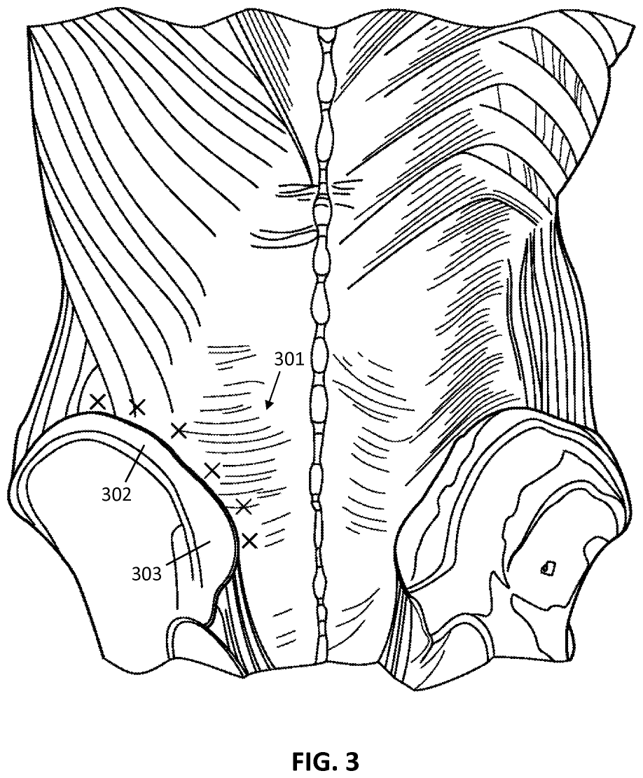

Any suitable palpation protocol may be used. For example, the palpation may be performed as follows: (1) beginning along the lower sacrum, the lateral edge of one side of the sacrum is palpated, from inferior (away from the head) to superior (toward the head), with palpation sites approximately ½″ to 1″ apart (See, FIG. 1 at [102]), (2) the area immediately lateral to the median crest of the same side of the sacrum is palpated from inferior to superior, with palpation sites approximately ½″ to 1″ apart (See, FIG. 1 at [101]), (3) the palpations described in (1)-(2) are repeated on the contralateral side of the sacrum; (4) the medial and lateral margins of the posterior superior iliac spine are palpated on each side from inferior to superior, with palpation sites approximately ½″ to 1″ apart (See, FIG. 3 at [303]; FIG. 2 at [202]); and (5) the superior and inferior margins of the posterior iliac crest on each side are palpated, proceeding up to the lateral aspect of the iliac crest or to the anterior superior iliac spine, with palpation sites approximately ½″ to 1″ apart (See, FIG. 3 at [302]; FIG. 2 at [204]). Exemplary sites for palpation on the left side of a subject are illustrated in FIGS. 1-3 and are shown on each of the figures as marked with an “X”. The order of palpating these sites may occur in any suitable order. For example, the beginning palpation at the anterior superior iliac spine and proceeding to the lower sacrum may help facilitate identification of the iliac crest in obese patients.

In some embodiments, areas of focal pain and/or tenderness identified by palpation are selected as injection sites, and the HA composition is injected into or immediately adjacent to the soft tissue attachments to these sites. The palpation may identify 1, 2, 3, 4, 5, 6, 7, 8, 9, 10, 15, 20, 25, or 30 injection sites, or a number of injection sites within a range bounded by any of the foregoing. In some embodiments, the one or more injections are made into 2-10 identified injection sites in a single treatment.

The composition may be administered using any suitable dosing scheme. For example, the composition may be administered daily, or once every 1, 2, 4, 6, 8, 10, 12, 24, or 36 weeks, or once within a range bounded by any of the foregoing. In some embodiments, the composition is administered once per week. In other embodiments, the composition is administered once every 4 to 8 weeks. In further embodiments, the composition is administered once per week during an initial treatment period and once every 4 to 8 weeks following completion of the initial treatment period. In some embodiments, the initial treatment period comprises two separate injection sessions, one week apart from each other. Prior to each administration, the palpation methods described herein may be repeated to identify sites for injection.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including.” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument, and human error in forming measurements, and the like. All percentages referring to amounts are by weight unless indicated otherwise.

No admission is made that any reference, including any non-patent or patent document cited in this specification, constitutes prior art. In particular, it will be understood that. unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

The Examples below demonstrate that HA injections into, or immediately adjacent to, the soft tissue attachments to the iliac crest, the posterior superior iliac spine, and/or the sacrum can be used to effectively treat patients with lower back, sacral, and/or buttocks pain. In the Examples, unless otherwise specified, pain severity was assessed using a visual analog numerical rating scale ranging from 0 (no pain) to 10 (worst pain possible), and hyaluronic acid (HA) injections (Hyalgan™, 10 mg/mL, MW: 500,000 to 730,000 Daltons in 2 mL pre-filled syringes) were administered using a 30 gauge, ½″ needle.

### Example 1

In July 2023, a 51 year-old female patient reported feet tingling and sacral pain, radiating to her buttocks bilaterally, and down her right thigh. The pain had persisted since 2020 (with significant worsening in the months preceding presentation) and was exacerbated by sitting or standing, and resolved with lying down. The patient previously had a vertebroplasty for an L1 compression fracture suffered in 2017, following which the pain had resolved. Physical exam revealed focal tenderness along the iliac crest, distinctly of the posterior superior iliac spine bilaterally. With compression applied over the anterior ilia while lying recumbent, pain was reproduced in her sacral area.

Prior to the initial visit, an MRI of her lumbar spine showed (1) degenerative changes of the lumbar spine, most advanced at L5-S1 where there is similar mild narrowing of the lateral recesses with possible contact of the descending S1 nerve roots without significant spinal canal or neural foraminal stenosis, (2) no significant spinal canal or neural foraminal stenosis at the additional levels, and (4) chronic superior endplate compression deformity of the L1 vertebral body with suggestion of prior vertebroplasty.

The patient was prescribed Norco for the ongoing back pain, and had received epidural steroid injections since 2020, with no benefit. A previous EMG (electromyography) and NCS (nerve conduction study) showed mild axonal sensorimotor polyneuropathy, but no evidence of a lumbar or sacral motor radiculopathy,

A pelvic CT scan performed between visits showed (1) no acute intrapelvic process, (2) spondyolisthesis with grade I retrolisthesis of L5 on S1, and (3) facet arthropathy, mild to moderate degree. A nuclear medicine bone showed changes consistent with the L1 compression fracture, but no other significant abnormalities.

On Sep. 21, 2023, the patient reported recurring lower back pain, with a typical severity of 7/10 (severe-to-very severe). Palpation identified focal tenderness along her posterior superior iliac spines bilaterally. For the reported back/sacral pain, a total of 4 mL (40 mg) of HA was injected as follows: 1 mL along the more inferior aspect of her left PSIS, 1 mL along the more superior aspect of her left PSIS, and 1 mL each along the inferior and superior aspect of her right PSIS.

On Sep. 28, 2023, the patient reported significant improvement in pain severity, which was now typically only 3/10 (mild-to-moderate) while walking. A second round of HA injections was administered into the soft tissues of the lower back as follows: 1 mL along the more inferior portion of the right PSIS; 1 mL along more superior portion of right PSIS; 2 mL along the posteromedial right iliac crest, just lateral to the PSIS; 1 mL along the inferior portion of left PSIS; 1 mL along more superior portion of left PSIS; and 2 mL along the posteromedial left iliac crest, just lateral to the left PSIS. In total, 8 mL (80 mg) of HA was injected.

On Oct. 24, 2023, the patient reported that lower back pain while ambulating was now essentially absent, although mild pain was present while lying flat and then extending her hips forward.

On Nov. 30, 2023, the patient reported the gradual return of the lower back pain, reaching a severity of 6/10 (average) and 9/10 (maximum) on the right side, and 6/10 (average and maximum) on the left side. Palpation was used to determine the location of the right PSIS and to identify focal tenderness along both the medial and lateral margins of the right PSIS. 4 mL (40 mg) of HA was injected near the right PSIS as follows: 1 mL into the soft tissues immediately adjacent to the inferomedial margin of the PSIS and 1 mL approximately 1″ superiorly toward the superomedial margin of the PSIS; 1 mL into the soft tissues immediately adjacent to the inferolateral margin of the right PSIS and 1 mL approximately 1″ superiorly toward the superolateral margin of the PSIS, Proceeding approximately 2″ to 3″ laterally along the posterior iliac crest from the previous injection site, 2 mL (20 mg) HA was injected into the fibro-osseous attachments of the lateral erector spinae or the quadratus lumborum to the iliac crest as follow: 1 mL into the soft tissues immediately adjacent to the superior margin of the iliac crest and 1 mL approximately 1″ laterally along the superior margin of the iliac crest. 2 mL (20 mg) HA was injected into the fibro-osseous attachments of the gluteus maximus and/or gluteus medius to the inferior margins of the iliac crest: 1 mL at each of two injection sites at the inferior margin of the iliac crest, at the same lateral location as the injections along the superior margin.

On Jan. 4, 2024, the patient reported a pain-free period of 3-4 weeks following the previous injections, similar to the pain-free period following the first round of two injection sessions (Sep. 21, 2023 and Sep. 28, 2023), but the lower back pain had returned by the time of the visit, with an average severity of 2/10 (mild) and maximum severity of 4/10 (moderate).

### Example 2

On Oct. 5, 2023, a 55 year-old female patient reported chronic, bilateral lower back pain, and more recent onset of pain in her feet that slowly spread to her lower leg, but mostly resides on the plantar surface of her feet. The lower back pain severity was 7/10 (between severe and very severe) at rest, and up to 9/10 (between very severe and worse pain possible) after standing all day. The patient had intact strength in lower extremities, with bilateral focal tenderness along the posterior superior iliac spines, extending laterally from the posterior superior iliac spines along the posterior iliac crest for several inches.

An MRI performed Oct. 10, 2023 showed: a slight wedge-shaped appearance of L1, appearing to be chronic; 2 mm retrolisthesis of L2 and L3, with 2 mm disc bulge; 1 mm retrolisthesis of L3 on L4, with 1.5 mm disc bulge; 4 mm anterolisthesis of L4 on L5; facet arthropathy contributing to mild left neural foraminal narrowing; mild multifactor spinal canal narrowing including 4 mm anterolisthesis of L5 on S1; and mild left greater than right neural foraminal narrowing.

On Oct. 5, 2023, after signing informed consent, she was treated for her chronic lower back pain with hyaluronic acid (HA) injections as follows: 4 injections along the left sacral attachments of the erector spinae (0.5 mL each); 4 injections along the right sacral attachment of the erector spinae (0.5 mL each); 4 injections (2 each on the superior and inferior aspects of the posterior superior iliac spine [PSIS]) along the left posteromedial iliac crest, primarily most medial along the PSIS (0.5 mL each); 4 injections (2 each on the superior and inferior aspects of the PSIS) along the right posteromedial iliac crest, primarily most medial along the PSIS (0.5 mL each). The injection locations were determined by findings of focal tenderness of the palpated sites illustrated in FIG. 1.

On Oct. 12, 2023, patient reported maximum lower back pain severity of 5-6/10 (severe). She was given a repeat injection of HA into her lower back as follows: 4 injections along the left sacral attachments of the erector spinae (0.5 mL each); 4 injections along the right sacral attachment of the erector spinae (0.5 mL each); 2 injections (1 each on the superior and inferior aspects of the PSIS) along the left posteromedial iliac crest, along the PSIS (1 mL each); and 2 injections (1 each on the superior and inferior aspects of the PSIS) along the right posteromedial iliac crest, along the PSIS (1 mL each). These injection locations were again determined by findings of focal tenderness of palpated sites illustrated in FIG. 1.

On Nov. 9, 2023, the patient reported average lower back pain of 2-3/10 (mild-to-moderate) for most of the day, but in the preceding days the pain severity had increased towards the end of her work shift, up to approximately 7/10 (severe-to-very severe).

On Dec. 7, 2023 the patient reported relatively stable lower back pain since the previous visit. with a typical severity of 2-3/10 in the morning/afternoon, reaching 6-7/10 in the late afternoons towards the end of her work shift. Prior to the first HA injections, the pain would start at 5-6/10 (moderately severe-to-severe) in the morning and gradually worsen throughout the day.

On Jan. 8, 2024, the patient reported that the pain recurrence beginning the week of Christmas, with a typical severity of 3/10 in the early morning, 5/10 at mid-morning when starting work, 7/10 at mid-day, and 8-9/10 at the end of work and into the evening. HA injections were administered as follows: 2 injections along superomedial left PSIS (1 mL each); 2 injections along inferolateral left PSIS (1 mL each); 2 injections along superomedial right PSIS (1 mL each); and 2 injections along inferolateral right PSIS (1 mL each). These injection locations were again determined by findings of focal tenderness of palpated sites illustrated in FIG. 1.

Beginning on Oct. 2, 2023, the patient was treated for peripheral neuropathy lidocaine/prilocaine 2.5% cream applied to her feet bilaterally. This local treatment was not effective. Not until Nov. 9, 2023, after confirming the effectiveness of the HA injections for her back pain, was pregabalin prescribed at a dose of 75 mg bid, which was helpful for the foot pain, but did not prevent the recurrence of lower back pain reported Jan. 8, 2024.

On Feb. 6, 2024, the patient reported significant lower back pain improvement following the previous HA injections, with pain severity of approximately 2/10 starting work in the morning. 5/10 in the middle the day, and 7/10 towards the end of the day and in the evening.

On Mar. 5, 2024, the patient reported a recurrence of lower back pain, also exacerbated by a recent fall. HA injections were administered as followed: 2 injections along superomedial left PSIS (1 mL each); 2 injections along inferolateral left PSIS (1 mL each); 2 injections along superomedial right PSIS (1 mL each); and 2 injections along inferolateral right PSIS (1 mL each). These injection locations were again determined by findings of focal tenderness of palpated sites illustrated in FIG. 1.

On May 7, 2024, the patient reported that the lower back pain had not recurred, with current severity of about 2/10 in morning and for most of day. No further injections were administered, and by Aug. 15, 2024, the pain had remained mild with an average severity of 2/10 for most of the day and night.

### Example 3

On Oct. 17, 2023, a 59 year-old female patient reported chronic lower back pain in the bilateral lower lumbar area, with no pain in the hips, thighs, legs or feet. The back pain began approximately 10 years prior and had been worsening gradually over time, with a current maximum severity of 7/10 (severe-to-very severe) on a daily basis. Examination revealed focal tenderness along her posterior iliac crest bilaterally, extending into the more medial aspect of the iliac crest on the left. An MRI of her lumbar spine performed on Apr. 13, 2023 showed minimal lumbar spondylosis, no levels of high-grade spinal canal stenosis, and mild foraminal narrowing at LA-5.

The patient reported that previous chiropractic treatment had exacerbated the pain, previous treatment with NSAIDs, muscle relaxants, and Norco (hydrocodone/acetaminophen combination) had provided no benefit, and an ongoing treatment with tramadol and cyclobenzaprine helped only marginally. She had previously tried NSAIDs, muscle relaxants, and Norco (hydrocodone/acetaminophen combination) without benefit.

The findings of an interventional pain management specialist earlier in 2023 were as follows: tenderness to left and right buttocks in lumbar region, no tenderness to left or right greater trochanter, tenderness to left and right sacroiliac joint, positive Faber's test on the left and right, and straight leg test on the left radiates left and straight leg raise on the right radiates right. Additional comments on the physical exam included a positive facet challenge test over the lumbar spine joints, and tenderness over sacroiliac joints. The patient was treated by interventional pain management with fluoroscopically guided medial branch blocks at L4, L5 and the sacral ala bilaterally, with no benefit from these injections. The patient was subsequently treated with a fluoroscopically guided lumbar epidural steroid injection at the L5-S1 interspace, and reported only two days of relief after this treatment. The patient declined further lumbar epidural steroid injections.

On Oct. 17, 2023, the patient was diagnosed with lower back pain with enthesopathy, and hyaluronic acid (HA) injections were administered as follows: 2 injections along the left posterior iliac crest (2 sites, 1 mL each); 2 injections along the left posteromedial iliac crest (PSIS; 2 sites, 1 mL each); and 4 injections along the right posterior iliac crest (4 sites, 1 mL each). These injection locations were determined by findings of focal tenderness of palpated sites illustrated in FIG. 1.

On Oct. 24, 2023, the patient reported the pain had decreased, with a daily maximum severity of 4-6/10 in the preceding days. On Oct. 26, 2023, the patient reported a maximum pain severity of 0 -1/10 in the preceding 24 hours at its worse. HA injections were administered as follows: 8 injections (0.5 mL each) along the left posterior iliac crest, with 4 sites along superior margin and 4 sites along inferior margin—extending to PSIS; and 8 injections (0.5 mL each) along the right posterior iliac crest, with 4 sites along superior margin and 4 sites along inferior margin—extending to PSIS.

On Nov. 27, 2023, the patient reported a pain-free period of approximately one week following the previous HA injection. While pain had begun to recur, with a maximum severity of 5-6/10 for several hours a day, the patient was pain-free for most of the day. The patient reported similar symptoms on Jan. 2, 2024. In comparison, the pain was constant prior to the HA.

On Jan. 15, 2024, the patient reported gradual recurrence of pain in the preceding 7 days, with a severity of 5-6/10 most of the day and a maximum severity of 8-9/10. HA injections, using palpatory guidance, were administered as follows: 8 injections (0.4 mL each) along the posterior iliac crest, with 4 sites along the superior margin and 4 sites along the inferior margin—extending to PSIS; and 8 injections (0.5 mL each) along the right posterior iliac crest, with sites along the superior margin and along the inferior margin—extending to PSIS.

On Mar. 15, 2024, the patient reported the back pain had improved from being constantly present to being pain free most of the day, though would still have pain that lasted a couple of hours per day. HA injections were administered as follows: 8 injections (0.5 mL each) along the left posterior iliac crest, with 4 sites along superior margin and 4 sites along inferior margin—extending to PSIS; and 8 injections (0.5 mL each) along the right posterior iliac crest, with 4 sites along superior margin and 4 sites along inferior margin—extending to PSIS.

On May 15, 2024, the patient reported general improvement following the previous injections, having back pain for only about an hour and a half a day, with many pain-free days. In the preceding several days, the lower back pain severity had started to increase, though it remained much less intense than prior to HA treatment. On May 21, 2024, HA injections were administered as follows: 8 injections (0.5 mL each) along the left posterior iliac crest, with 4 sites along superior margin and 4 sites along inferior margin—extending to PSIS; and 8 injections (0.5 mL each) along the right posterior iliac crest, with 4 sites along superior margin and 4 sites along inferior margin—extending to PSIS.

### Example 4

A 47-year-old male reported chronic low back pain, which had persisted for the past year after suffering a fall. X-rays and MRIs of the patient's lumbar spine showed no evidence of fracture. The pain was present in his left lower back, in a focal region along the superior margin of his gluteus maximus. The daily average pain was 7/10 in the several months prior to the appointment, reaching a maximum severity of approximately 9/10. The patient was not experiencing any leg pain or tingling, and prior treatment with cyclobenzaprine and ibuprofen was without benefit. Examination revealed focal, distinct tenderness at his left posterior superior iliac spine (PSIS), on the lateral margins where his gluteus maximus attaches. Hyaluronic acid injections were administered in the form of Hyalgan (20 mg/ml) along the lateral margin of the left PSIS (two injections; 1 mL each). Upon re-evaluation eleven days later, the patient reported an average pain severity of 2/10 or less, with a maximum severity of 2/10, including while working and exercising. Examination revealed only mild tenderness along the lateral margin of his left PSIS, where his gluteus maximus attaches. He received subsequent injections on this visit: again two injections of 1 mL each along the lateral margins of his left PSIS. He was re-evaluated 30 days later, and stated that—with the exception of a few days of moderate pain caused by a work-related back strain—the pain remained well-controlled, with an average pain severity of 1-2/10.

### Example 5 (Prophetic)

A 64-year-old male patient will report focal pain in the buttock and sacral area, without radiation to the lower leg, the pain having persisted since a motor vehicle accident more than three months prior. On exam he will have intact strength and reflexes to his lower extremities, no tenderness to palpation of his left greater trochanteric region or iliac crest, but focal tenderness along the left lateral edge of his sacrum. X-rays of his hip will be within normal limits, with no evidence of hip arthritis, sacroiliac joint arthritis, or other pathology discernible by X-ray. There will be distinct tenderness to palpation all along the left lateral edge of his sacrum. He will be administered hyaluronic acid by injection, at a concentration of 10mg/ml, in doses of 0.5 mL to four separate sites along the lateral edge of his sacrum. This will be repeated one week later, and the pain will be significantly improved for two months.

### REFERENCES

1. Hartvigsen et al., What low back pain is and why we need to pay attention, Lancet 2018 June 9; 391(10137):2356-2367.
2. Karl H W et al., Superior and middle cluneal nerve entrapment: a cause of low back and radicular pain, Pain Physician 2022; 25:E503-521.
3. Morimote et al., Surgical treatment of superior cluneal nerve entrapment neuropathy, Journal of Neurosurgery Spine 2013; 19:71-75.
4. Hansson P. Neuropathic pain: clinical characteristics and diagnostic workup, European Journal of Pain, 2002; 6 Supplement A:47-50.
5. Systemic Pharmacologic Therapies for Low Back Pain: A Systematic Review for an American College of Physicians Clinical Practice Guideline
6. Collee G. et al., Iliac crest pain syndrome in low back pain: frequency and features, Journal of Rheumatology 1991; 18(7): 1064-7,
7. Njoo K. et al., Interobserver agreement on the iliac crest pain syndrome in general practice, Journal of Rheumatology 1995; 22(8): 1532-5; Collee G. et al., Iliac crest pain syndrome in low back pain. A double blind, randomized study of local injection therapy, Journal of Rheumatology 1991; 18(7): 1060-3.
8. Naim F. et al., Treatment of the chronic iliolumbar syndrome by infiltration of the iliolumbar ligament, Western Journal of Medicine 1982; 136(4): 372-4,
9. Nayak B. et al., Recovering from nonspecific low back pain despair: Ultrasound-guided intervention in iliolumbar syndrome, The Indian journal of radiology & imaging 2020; 30(4): 448-52).
10. Liu I. et al., Ultrasonographic diagnosis and guided treatment of erector spinae aponeurosis enthesopathy, Medical ultrasonography 2022; 24(1): 120-21.
11. Wilkinson H, Injection therapy for enthesopathies causing axial spine pain and the “failed back syndrome”: a single blinded, randomized and cross-over study Pain Physician 2005; 8:167-173
12. Neumann et al., High molecular weight hyaluronic acid inhibits advanced glycation endproduct-induced NF-KB activation and cytokine expression, FEBS Letters 1 999; 453:283-87.
13. Agostini F et al., Effects of hyaluronic acid injections on pain and functioning in patients affected by tendinopathies: A narrative review, Journal of Back and Musculoskeletal Rehabilitation, 2022; 35:949-961.
14. Nesporova K. et al., Injecting hyaluronan in the thoracolumbar fascia: A model study, International Journal of Biological Macromolecules, 2023; 253(Pt 3): 126879.
15. Campa J A, Cross-Linked Hyaluronic Acid for the Management of Neuropathic Pelvic Pain, Practical Pain Management, 2018; 18(5)
16. Amirdelfan K. et al., Allogeneic mesenchymal precursor cells treatment for chronic low back pain associated with degenerative disc disease: a prospective randomized, placebo-controlled 36-month study of safety and efficacy, The Spine Journal, 2021; 21(2): 212-230.

17. Willard F H et al., The thoracolumbar fascia: anatomy, function and clinical considerations, J Anat. 2012 Dec; 221(6):507-36).
18. Collee G. et al., Iliac crest pain syndrome in low back pain. A double blind, randomized study of local injection therapy, Journal of Rheumatology 1991; 18(7): 1060-3.

### EMBODIMENTS

1. A method of treating pain in a subject, comprising administering to the subject a composition comprising (a) a therapeutically effective amount of hyaluronic acid and (b) a pharmaceutically acceptable carrier, wherein the subject is experiencing pain in the lower back. buttocks, and/or sacral region, and wherein the composition is administered by one or more injections.
2. The method of embodiment 1, wherein the pain is nociceptive pain.
3. The method of embodiment 1 or 2, wherein the one or more injections are made into, or immediately adjacent to, soft tissue attachments to the subject's iliac crest, posterior superior iliac spine, and/or sacrum.
4. The method of any one of embodiments 1-3, wherein the one or more injections are made into the soft tissue attachments within about ½″ to about 1″ from the iliac crest, posterior superior iliac spine, and/or sacrum.
5. The method of any one of embodiments 1-4, wherein the composition is administered by two or more injections along the subject's iliac crest, posterior superior iliac spine, and/or sacrum, and wherein the two or more injections are spaced at an interval of about ¼″ to about 4″.
6. The method of embodiment 5, wherein the two or more injections are spaced at an interval of about ½″ to about 1″.
7. The method of any one of embodiments 1-6, further comprising identifying one or more injection sites for the one or more injections, wherein the identifying comprises palpating the iliac crest, posterior superior iliac spine, and/or sacrum and selecting areas of pain or tenderness on palpating for injection.
8. The method of embodiment 6 or 7, wherein the one or more injections are made into 2-30 injection sites.
9. The method of embodiment 8, wherein the one or more injections are made into 2-10 injection sites
10. The method of any one of embodiments 1-9, wherein the hyaluronic acid has a molecular weight of at least 500 kilodaltons (kDa).
11. The method of embodiment 10, wherein the hyaluronic acid has a molecular weight of about 500 kDa to about 730 kDa.
12. The method of any one of embodiments 1-11, wherein the hyaluronic acid is present in the composition at a concentration of 5 mg/mL to 20 mg/mL.
13. The method of embodiment 12, wherein the hyaluronic acid is present in the composition at a concentration of about 10 mg/mL.
14. The method of any one of embodiments 1-13, wherein each injection of the one or more injections delivers 5 mg or more of the hyaluronic acid.
15. The method of embodiment 14, wherein each injection of the one or more injections delivers 5 mg to 10 mg of the hyaluronic acid.
16. The method of any one of embodiments 1-15, wherein the composition is administered once per week.
17. The method of any one of embodiments 1-15, wherein the composition is administered once every four to eight weeks.
18. The method of any one of embodiments 1-15, wherein the composition is administered (a) once per week during an initial treatment period and (b) once every four to eight weeks following completion of the initial treatment period.
19. The method of embodiment 18, wherein the initial treatment period comprises two separate injection sessions, the injection sessions separated by about one week.
20. The method of any one of embodiments 1-19, wherein the one or more injections are made into one or more sites at locations [101], [102], [202], [204], [302], and/or [303], as shown in FIGS. 1-3.

1. A method of treating pain in a subject, comprising administering to the subject a composition comprising (a) a therapeutically effective amount of hyaluronic acid and (b) a pharmaceutically acceptable carrier, wherein the subject is experiencing nociceptive pain in one or more of the lower back, buttocks, or sacral region, and wherein the composition is administered by one or more injections made into, or immediately adjacent to, soft tissue attachments to one or more of the subject's iliac crest, posterior superior iliac spine, or sacrum, and wherein the injections are not made into or immediately adjacent to a tendon, an osteotendinous junction, or a musculotendinous junction.

2. The method of claim 1, wherein the one or more injections are made into the soft tissue attachments within about ½″ to about 1″ from the iliac crest, posterior superior iliac spine, and/or sacrum.

3. The method of claim 1, wherein the composition is administered by two or more injections along the subject's iliac crest, posterior superior iliac spine, and/or sacrum, and wherein the two or more injections are spaced at an interval of about ¼″ to about 4″.

4. The method of claim 1, wherein the two or more injections are spaced at an interval of about ½″ to about 1″.

5. The method of claim 1, further comprising identifying one or more injection sites for the one or more injections, wherein the identifying comprises palpating the iliac crest, posterior superior iliac spine, and/or sacrum and selecting areas of pain or tenderness on palpating for injection.

6. The method of claim 1, wherein the one or more injections are made into 2-30 injection sites.

7. The method of claim 1, wherein the one or more injections are made into 2-10 injection sites.

8. The method of claim 1, wherein the hyaluronic acid has an average molecular weight of 100 kilodaltons (kDa) to 10,000 kDa.

9. The method of claim 8, wherein the hyaluronic acid has a molecular weight range of 500 kDa to 800 kDa.

10. The method of claim 1, wherein the hyaluronic acid is present in the composition at a concentration of 5 mg/mL to 20 mg/mL.

11. The method of claim 10, wherein the hyaluronic acid is present in the composition at a concentration of about 10 mg/mL.

12. The method of claim 1, wherein each injection of the one or more injections delivers 5 mg to 10 mg of the hyaluronic acid.

13. The method of claim 1, wherein each injection of the one or more injections delivers 10 mg to 20 mg of the hyaluronic acid.

14. The method of claim 1, wherein the composition is administered once per week.

15. The method of claim 1, wherein the composition is administered once every four to eight weeks.

16. The method of claim 1, wherein the composition is administered (a) once per week during an initial treatment period and (b) once every four to eight weeks following completion of the initial treatment period.

17. The method of claim 16, wherein the initial treatment period comprises two separate injection sessions, the injection sessions separated by about one week.

18. The method of claim 1, wherein the composition is administered every 8-24 weeks.

19. The method of claim 1, wherein the one or more injections are made into one or more sites at locations [101], [102], [202], [204], [302], and/or [303], as shown in FIGS. 1-3.
